(19) United States
(12) Patent Application Publication
(10) Pub. No.: US 2024/0139112 A1
(43) Pub. Date: May 2, 2024

(54) EXOSOMES COMPRISING IL-35 OR IL-27 AND USES THEREOF

(71) Applicant: The United States of America,as represented by the Secretary,Department of Health and Human Services, Bethesda, MD (US)

(72) Inventors: Charles E. Egwuagu, Silver Spring, MD (US); Minkyung Kang, Rockville, MD (US)

(73) Assignee: The United States of America,as represented by the Secretary,Department of Health and Human Services, Bethesda, MD (US)

(21) Appl. No.: 18/271,583

(22) PCT Filed: May 28, 2021
(86) PCT No.: PCT/US2021/034778

§ 371 Date: Jul. 10, 2023

Related U.S. Application Data
(60) Provisional application No. 63/135,833, filed on Jan. 11, 2021.

(51) Int. Cl. A61K 9/50; A61K 38/20; A61P 37/06; C12N 5/0781

(52) U.S. Cl. CPC A61K 9/5068; A61K 38/20; A61P 37/06; C12N 5/0635

(57) ABSTRACT

In an embodiment, the invention provides an isolated population of exosomes comprising interleukin-27 (IL-27) or interleukin-35 (IL-35). In an embodiment, the invention also provides a method of preparing a population of exosomes comprising interleukin-27 (IL-27), the method comprising: (a) isolating CD19+B2 cells or B1a cells; (b) activating the isolated cells with a LPS or a BCR agonist to provide activated cells; and (c) isolating exosomes secreted from the activated cells. In an embodiment, the invention also provides a method of preparing a population of exosomes comprising interleukin-35 (IL-35), the method comprising: (a) isolating CD138+plasma cells; (b) activating the isolated cells with a LPS or a BCR agonist to provide activated cells; and (c) isolating exosomes secreted from the activated cells. Additional embodiments of the invention are as described.

### CROSS REFERENCE TO RELATED APPLICATION

This patent application claims the benefit of U.S. Provisional Patent Application No. 63/135,833, filed Jan. 11, 2021, which is incorporated by reference in its entirety herein.

### STATEMENT REGARDING FEDERALLY SPONSORED RESEARCH OR DEVELOPMENT

This invention was made with Government support under project numbers EY000350-21 and EY000372-20 by the National Institutes of Health, National Eye Institute. The Government has certain rights in the invention.

### BACKGROUND OF THE INVENTION

The central nervous system (CNS) is an immune-privileged site comprised of the brain, spinal cord, and the ocular retina. Its intricate and highly vulnerable physiology is shielded from potentially pathogenic inflammatory cells by the blood-brain-barrier (BBB) or the blood-ocular-barrier (BOB). Although resident ocular microglial cells or epithelial cells of the choroid plexus that constitutively secrete immunosuppressive cytokines contribute to the maintenance of immune privilege of the eye, brain, and spinal cord, lymphocytes bearing antigen-receptors specific to oligodendrocytes or retinal proteins do breach the BBB or BOB during neuroinflammatory diseases and attack and destroy neurons and photoreceptor cells.

Thus, inflammation in the CNS presents unique challenges, and there is a need to avoid collateral damage that may compromise functional integrity of the retina or brain when treating or preventing a pathology involving the CNS.

### BRIEF SUMMARY OF THE INVENTION

In an embodiment, the invention provides an isolated population of exosomes comprising interleukin-27 (IL-27) or interleukin-35 (IL-35).

In an embodiment, the invention also provides a method of preparing a population of exosomes comprising interleukin-27 (IL-27), the method comprising: (a) isolating CD19+B2 cells or B1a cells; (b) activating the isolated cells with a LPS or a BCR agonist to provide activated cells; and (c) isolating exosomes secreted from the activated cells.

In an embodiment, the invention also provides a method of preparing a population of exosomes comprising interleukin-35 (IL-35), the method comprising: (a) isolating CD138+plasma cells; (b) activating the isolated cells with a LPS or a BCR agonist to provide activated cells; and (c) isolating exosomes secreted from the activated cells.

Additional embodiments of the invention are as described herein.

### BRIEF DESCRIPTION OF THE DRAWINGS

Results shown in the figures as described below are in accordance with aspects of the invention.

FIG. 1A are two sets of graphs and photos that present results of purification, characterization, and quantification of exosomes derived from activated B cells that do not produce IL-35 (Naïve-Exosomes; top set of graph and photo) or IL-35-secreting regulatory B cells (i35-Exosomes; bottom set of graph and photo). Size distribution analysis of exosome samples analyzed by Nanoparticle Tracking Analysis (NTA). Mean±SEM of three independent experiments is shown.

FIGS. 1B and 1C are bar graphs that present quantification of exosomes released in unstimulated (labelled “1”) and stimulated (labelled “2”) B cell cultures by use of the Exosome Quantitation Assay (FIG. 1B) and determination of amounts of IL-35 contained in 2×10 10 Naïve-Exosomes (labelled “1”) or i35-Exosomes (labelled “2”) (n=6) by ELISA (FIG. 1C).

FIG. 1D presents Western blot analysis of exosomal markers (HSP70 and CD63) expressed by exosomes derived from i35-Breg cells (right lane, 2) or exosomes from B cells that do not produce IL-35 (Naïve-Exosomes; left lane, 1).

FIG. 1E presents immunoprecipitation/Western blots that show lysates derived from Naïve-Exosomes (left lane, 1) or i35-Exosomes (right lane, 2) that were subjected to immunoprecipitation/Western blot analysis using antibodies specific to Ebi3 or p35 (left panel) and i35-Exosomes subjected to immunoprecipitation/Western blot analysis using antibodies specific to Ebi3 or p35 or mouse-IgG (right panel).

FIGS. 1F and 1G are bar graphs showing results of CD4+ T cells that were stimulated in vitro for 3 days in culture medium containing anti-CD3/CD28 Abs and Naïve-Exosomes (labelled “1”) or i35-Exosomes (labelled “2”). Secretion of IL-2 (FIG. 1F) or IFN-γ (FIG. 1G) in the supernatants was detected by ELISA (n=6). Results represent three independent studies, ****p<0.0001.

FIG. 1H is a series of graphs that present results after CD4+ T cells were stimulated with anti-CD3/CD28 Abs for 4 days under non-polarizing condition in culture medium containing Naïve-Exosomes or i35-Exosomes (20 μg).

FIG. 11 is a bar graph showing the effect of Naïve-Exosomes or i35-Exosomes on lymphocyte proliferation assessed by the CFSE dilution assay. Results represent three independent studies, **p<0.01, ***p<0.001.

FIG. 2A presents the timeline scheme used for exosome treatment as described in Example 1.

FIG. 2B presents fundus images of the retina that were taken at days 15 and 17 after EAU induction using an otoendoscopic imaging system, along with corresponding histograms. Compared to mice treated with i35-Exosomes, fundus images of mice treated with PBS revealed more severe ocular inflammation characterized by significant blurring of the optic disc margins and enlarged juxtapupillary area (black arrow), retinal vasculitis (circled arrows), yellow-whitish retinal and choroidal infiltrates (white arrow). Clinical scores and assessment of disease severity were based on changes at the optic nerve disc or retinal vessels and retinal and choroidal infiltrates. Histograms show the clinical scores (n=14). Results represent three independent studies, ***p<0.001, ****p<0.0001

FIG. 2C presents histological images. Eyes show very severe EAU in mice treated with PBS as characterized by the development of massive retinal in-folding (*), a hallmark feature of severe uveitis. H&E histological sections: Scale bar, 100 μm. V, vitreous; GCL, ganglion cell layer; INL, inner nuclear layer; ONL, outer nuclear layer; RPE/CH retinal pigmented epithelial and choroid.

FIG. 2D presents representative OCT images showing marked decrease of inflammatory cells (white arrows) in the vitreous and optic nerve head of mice treated with i35-Exosomes. Circled arrows show retinal-folds that represent hallmark of severe uveitis.

FIG. 2E are bar graphs that present ERG analysis of the retina on day-17 after EAU induction. The averages of light- or dark-adapted ERG a-wave or b-wave amplitudes are plotted as a function of flash luminance and values are means±SEM. Data are presented as the mean±SEM of at least three determinations. Results represent three independent studies. *p<0.05, **p<0.01, ****p<0.0001.

FIG. 3A are graphs that present analysis of CD4+ lymphocytes in the eye, spleen, or lymph nodes of mice treated with PBS or i35-Exosomes by the intracellular cytokine assay (n=14). Data are presented as mean±SEM of three replicates, and results represent three independent studies, **p<0.01, ***p<0.001.

FIGS. 3B and 3C are graphs that present analysis of CD4+ T cells, in the eye of mice treated with PBS or i35-Exosomes, by the intracellular cytokine assay (n=14) for IFNγ (FIG. 3B) and Fox3p and p35/Ebi3 (FIG. 3C). Data are presented as mean±SEM of three replicates, and results represent three independent studies, *p<0.05, **p<0.01.

FIG. 4 are bar graphs showing CD19⁺CD5⁺ B1a cells were sorted from peritoneal cavity and stimulated in culture with LPS or BCR (IgM/anti-CD40) for 72 hrs. Percentage of CD19⁺CD5⁺CD23⁻ or CD19⁺CD5⁺CD23⁻ CD81⁺ B1a cells expressing p28, Ebi3 or IL-27 (p28+Ebi3) were determined by intracellular cytokine staining assay. Y-axis (Counts) indicate percentage of B1a cells expressing p28, Ebi3 or IL-27 (p28+Ebi3).

FIG. 5 is a bar graph showing more i27-Exosomes were released by activated peritoneal cavity B1a cells compared to CD19+ B-2 cells derived from the spleen.

FIG. 6 is a bar graph showing 50 μg i27-Exosomes derived from mouse peritoneal cavity B1a cells contain 100 pg/mL of Interleukin 27 (IL-27)

FIG. 7 is a series of graphs that present flow cytometry plots and bar graphs showing B-1 cells in the spleen also secrete IL-27 and inhibit T cell proliferation.

FIG. 8 is a graph showing i27-Exosomes suppressed experimental autoimmune uveitis (EAU).

FIGS. 9A and 9B are flow cytometry plots (FIG. 9A) and bar graphs (FIG. 9B) showing i27-Exosomes suppressed EAU by inhibiting pro-inflammatory responses (IL-17 and IFN-γ) while inducing the expansion of regulatory T cells.

FIGS. 10A and 10B are flow cytometry plots (FIG. 10A) and bar graphs (FIG. 10B) showing i27-exosomes ameliorated uveitis by suppressing proliferation of uveitogenic T cells.

### DETAILED DESCRIPTION OF THE INVENTION

B cells (or B-lymphocytes) are lymphocytes which differentiate into plasma cells that secrete antibodies. Immature B cells are produced in the bone marrow of most mammals. After reaching the IgM⁺ immature stage in the bone marrow, these immature B cells migrate to the spleen, where they are called transitional B cells, and some of these cells eventually differentiate into mature B lymphocytes. B cell development occurs through several stages, with each stage representing a change in the genome content of antibody genes.

Mature B cells can be classified as either plasma B cells (also known as plasma cells, plasmocytes, or effector B cells) or memory B cells. Plasma B cells are large B cells that have been exposed to antigen and produce and secrete large amounts of antibodies. Plasma B cells are short-lived and undergo apoptosis when the antigen that induced a particular immune response is eliminated. In contrast, memory B cells are long-lived stimulated B cells that are primed for rapid response to a repeated exposure of a priming antigen. Memory B cells are generated in lymphoid tissue after B cell activation/proliferation and reside in the bone marrow, lymph nodes, and spleen.

Each B cell has a unique receptor protein on its surface that will bind to one particular antigen, which is referred to as the B cell receptor (BCR). The BCR is a membrane-bound immunoglobulin, which allows the distinction of B cells from other types of lymphocytes, and is the main protein involved in B cell activation. Once a B cell encounters its cognate antigen and receives an additional signal from a T helper cell, the B cell can further differentiate into either a plasma B cell or a memory B cell. The B cell may differentiate into a plasma or memory B cell directly, or the B cell may undergo intermediate differentiation steps, called germinal center reactions, in which a B cell undergoes somatic hypermutation of the variable region of an immunoglobulin gene, and possibly class switching. Other functions of B cells include antigen presentation, cytokine production, and lymphoid tissue organization.

Certain B cells can suppress autoimmune diseases through production of anti-inflammatory cytokines such as IL-10, IL-35, or TGFβ, alone or in combination with inhibitory cell-surface receptors (these, e.g., being regulatory B cells of B2-lymphocyte lineage). Regulatory B cells (Bregs) that produce IL-35 (i35-Bregs) suppress encephalomyelitis and uveitis. Bregs of, e.g., B-1a lineage, that produce and secrete interleukin-27 (i27-Bregs) can also suppress autoimmune disease. Without wishing to be bound by any theory, Bregs inhibit Th1 and Th17 T cells and expand regulatory T cells (Tregs).

Interleukin-35 (IL-35) is a member of the IL-12 family of heterodimeric cytokines and is composed of Ebi3, a 13 chain subunit encoded by the Epstein-Barr virus (EBV)-induced gene 3 (also known as IL27b), and the IL12p35 a subunit encoded by IL-12a. IL-35 is produced by regulatory T cells and is involved in the immunosuppressive activities of Tregs.

Interleukin-27 (IL-27) is a member of the IL-12 cytokine family. IL-27 is a heterodimeric cytokine that is composed of two distinct protein subunits of Ebi3 and IL-27p28. IL-27 is expressed by cells and interacts with the IL-27 receptor (IL-27R). IL-27R consists of two proteins, IL-27a (IL-27 alpha) and gp130. IL-27 induces differentiation of the diverse populations of T cells in the immune system. Natural activation of B-1a regulatory cells upon inflammatory stimuli triggers IL-27 production and the coincident exodus of i27-Bregs to the spleen where they reprogram conventional lymphocytes to acquire immune-regulatory functions.

One difficulty of using Bregs for therapy is dosing because biologically active IL-35 and IL-27 are each a weakly associated heterodimer that readily dissociates, thereby making it difficult to ascertain the bioavailability of the IL-35 or IL-27 secreted by Bregs.

It has been unexpectedly and surprisingly found that i35-Bregs and i27-Bregs secrete exosomes that contain IL-35 (i35-Exosomes) and IL-27 (i27-Exosomes), respectively. Generally, exosomes are secreted by immune cells including lymphocytes. Exosomes contain proteins, lipids, nucleotides, miRNAs, and mRNAs, and their functions can vary depending on the cell of origin and its physiological state. Exosomes are nanosized vesicles of 30-150 nm (for example 30 nm, 40 nm, 50 nm, 60 nm, 70 nm, 80 nm, 90 nm, 100 nm, 110 nm, 120 nm, 130 nm, 140 nm, or 150 nm) that can cross the BBB or BOB, and deliver their cargo into the CNS.

In an embodiment, the invention provides an isolated population of exosomes comprising interleukin-27 (IL-27) or interleukin-35 (IL-35). In an embodiment, the invention provides an isolated population of exosomes comprising IL-27. In an embodiment, the invention provides an isolated population of exosomes comprising IL-35. In an embodiment, the invention provides an isolated population of exosomes comprising both IL-27 and IL-35.

The exosomes of the invention may induce expression of inhibitory receptors lymphocyte-activation gene 3 (LAG-3), programmed cell death protein 1 (PD-1), and C—X—C chemokine receptor type 4 (CXCR4) on surface of target cells. LAG-3 (or cluster of differentiation 223 (CD223)) is a protein encoded by the LAG3 gene in humans. LAG3 is an immune checkpoint receptor. PD-1 (or cluster of differentiation 279 (CD279)) is a protein encoded by the PDCD1 gene in humans. PD-1 is also an immune checkpoint receptor. PD-1 promotes apoptosis of antigen-specific T cells and reduces apoptosis in regulatory T cells (anti-inflammatory, suppressive T cells). CXCR4 (or fusin or cluster of differentiation 184 (CD184)) is a protein encoded by the CXCR4 gene in humans. CXCR4 is an alpha-chemokine receptor specific for stromal-derived-factor-1 (SDF-1 or CXCL12), a molecule with chemotactic activity for lymphocytes.

The exosomes may also induce cell surface expression of inhibitory receptor glucocorticoid-induced TNFR-related protein (GITR or tumor necrosis factor receptor superfamily member 18 (TNFRSF18) or activation-inducible TNFR family receptor (AITR)). GITR is a protein encoded by the TNFRSF18 gene in humans. GITR has been shown to have increased expression upon T cell activation. The exosomes may also induce cell surface expression of inhibitory receptor OX40 (or tumor necrosis factor receptor superfamily member 4 (TNFRSF4) or cluster of differentiation 134 (CD134)). OX40 is a protein encoded by the TNFRSF4 gene in humans. OX40 is not constitutively expressed on resting naïve T cells. The exosomes may also induce cell surface expression of inhibitory receptor cytotoxic T-lymphocyte-associated protein 4 (CTLA4 or cluster of differentiation 152 (CD152)). CTLA4 is a protein encoded by the CTLA4 gene in humans. CTLA4 is an immune checkpoint and downregulates immune responses. CTLA4 is constitutively expressed in regulatory T cells but only upregulated in conventional T cells after activation.

The exosomes can be from a cell or cells of a mammal. The term “mammal” includes, but is not limited to, the order Rodentia, such as mice, and the order Logomorpha, such as rabbits, the order Carnivora, including Felines (cats) and Canines (dogs), the order Artiodactyla, including Bovines (cows) and Swines (pigs), the order Perssodactyla, including Equines (horses), Primates, Ceboids, or Simioids (monkeys), and the order Anthropoids (humans and apes). More preferably, the exosomes are of a cell or cells from a human.

In an embodiment, the invention also provides a method of preparing a population of exosomes comprising interleukin-27 (IL-27), the method comprising: (a) isolating CD19+B2 cells or B1a cells; (b) activating the isolated cells with a LPS or a BCR agonist to provide activated cells; and (c) isolating exosomes secreted from the activated cells. The CD19+B2 cells or B1a cells can optionally be stimulated with IL-27. In an embodiment, the invention also provides a method of preparing a population of exosomes comprising interleukin-35 (IL-35), the method comprising: (a) isolating CD138+plasma cells; (b) activating the isolated cells with a LPS or a BCR agonist to provide activated cells; and (c) isolating exosomes secreted from the activated cells. The CD138+plasma cells can optionally be stimulated with IL-27.

The source mammal tissue or fluid sample can be from any suitable source, such as mammal peripheral lymphoid tissue, mammal cord blood, mammal peritoneal fluid, mammal bone marrow, induced pluripotent cells (iPSC), or any other sample containing CD19+B2, CD138+, or B-1a cells. In at least some embodiments, the use of peritoneal fluid or cord blood as the sample may be desirable because these sources typically have a higher percentage of B-1a cells than other samples (e.g., peripheral lymphoid tissue). In some embodiments, the preferred source of the tissue or fluid may be from the donor subject that will be treated with the population of exosomes of the invention. B1a cells originate from fetal tissues, mainly inhabit the peritoneal and pleural cavities, are larger than conventional B cells (B2), and are long-lived and self-renewing innate-like B cells. B1a cells are a major source of IL-10, inhibiting the progression of both innate and adaptive immune responses, preventing tissue damage.

Any suitable cell culture media that can support the growth of the cells can be used. For example, Roswell Park Memorial Institute medium (RPMI 1640) culture medium can be used.

The cultured cells can be exposed to a lipopolysaccharide (LPS) or a BCR agonist or a Toll-like receptor (TLR) agonist. Any suitable BCR agonist or a TLR agonist that can activate the cells can be used. Examples of BCR agonists include anti-CD40 and anti-IgM antibodies. Examples of TLR agonists include TLR9 and TLR4 agonists. As is the case for all lymphocytes, the cells should be activated to elicit biological activity. CD40 is a costimulatory protein found on antigen presenting cells and is involved in B cell activation following interaction of the B cell receptor with an antibody to IgM.

As used herein, the terms “Toll-like receptor” and “TLR” refer to any member of a family of highly-conserved mammalian proteins which recognize pathogen-associated molecular patterns and act as key signaling elements in innate immunity. TLR polypeptides share a characteristic structure that includes an extracellular domain that has leucine-rich repeats, a transmembrane domain, and an intracellular domain that is involved in TLR signaling.

The terms “Toll-like receptor 4” and “TLR4” refer to nucleic acids or polypeptides sharing at least 70%, 80%, 90%, 95%, 96%, 97%, 98%, 99%, or more sequence identity to a publicly-available TLR4 sequence. A suitable TLR4 agonist is LPS.

The terms “Toll-like receptor 9” and “TLR9” refer to nucleic acids or polypeptides sharing at least 70%, 80%, 90%, 95%, 96%, 97%, 98%, 99%, or more sequence identity to a publicly-available TLR9 sequence. Suitable TLR9 agonists are oligonucleotides containing CpG motifs (CpG ODNs).

The IL-27 or IL-35 protein optionally used for stimulation can be native IL-27 or IL-35 protein that is isolated from cells which naturally produce the protein. In this embodiment, the IL-27 or IL-35 protein preferably is isolated from a mammal (e.g., a human or a mouse). Alternatively, the protein can be a recombinant IL-27 or IL-35 protein generated using routine molecular biology techniques. A recombinant IL-27 or IL-35 protein can contain all or a portion of a native IL-27/IL-35 protein from a human or a mouse. For example, a recombinant protein can contain an entire native human protein or an entire native mouse protein. In another embodiment, a recombinant IL-27 or IL-35 protein can contain a portion of a native protein from a human and a portion of a native protein from a mouse (i.e., a “chimeric” protein). One of ordinary skill in the art will appreciate that a recombinant IL-27 or IL-35 protein can contain other elements that optimize the expression and/or stability of the IL-27 or IL-35 protein, e.g., in B-cells. In an embodiment, the IL-27 protein is a recombinant fusion protein comprising IL-2′7p28 and an Epstein-Barr virus (EBV)-induced gene 3 (Ebi3) protein. In an embodiment, the IL-35 protein is a recombinant fusion protein comprising an IL-12p35 a subunit protein and an Epstein-Barr virus (EBV)-induced gene 3 (Ebi3) protein.

The inventive methods are useful for the treatment of a disease in a mammal. The treatment may result in desirable suppression of the immune system.

The inventive methods are useful for the treatment, suppression, or prevention of graft-versus-host disease (GVHD). Patients can receive a solid organ or allogeneic bone marrow or hematopoietic stem cell transplant. In order to prevent or reduce the severity of GVHD, the population of exosomes of the invention are administered to a mammal before the mammal receives an allogeneic transplant. Alternatively, GVHD can be prevented or suppressed by mixing the population of exosomes of the invention with a transplant material to form a transplant mixture, and then administering the transplant mixture to the mammal. In this regard, the transplant material can include allogeneic lymphocytes. In an embodiment, the transplanted cells are cells (e.g., heart cells, pancreatic cells, retinal cells) derived from iPS cells.

The population of exosomes of the invention can be mixed with the transplant material ex vivo. “Ex vivo” refers to methods conducted within or on cells or tissue in an artificial environment outside an organism with minimum alteration of natural conditions. In contrast, the term “in vivo” refers to a method that is conducted within living organisms in their normal, intact state, while an “in vitro” method is conducted using components of an organism that have been isolated from its usual biological context.

The population of exosomes can be administered in the form of a pharmaceutically acceptable (e.g., physiologically acceptable) composition. The composition may comprise a carrier, preferably a pharmaceutically (e.g., physiologically acceptable) carrier, and the population of exosomes. Any suitable carrier can be used within the context of the invention, and many such carriers are known in the art. The choice of carrier will be determined, in part, by the particular site to which the composition may be administered and the particular method used to administer the composition. The composition optionally can be sterile. The composition can be frozen or lyophilized for storage and reconstituted in a suitable sterile carrier prior to use. The compositions can be generated in accordance with conventional techniques known in the art.

The population of exosomes can be administered to a mammal (as earlier defined herein). Preferably the mammal is a mouse or a human.

The invention provides a method of suppressing the immune system in a mammal, which method comprises administering the population of exosomes of the invention to a mammal in need thereof, thereby suppressing the immune system in the mammal. Thus, the invention provides for a method of suppressing autoimmunity in a mammal comprising administering an isolated population of exosomes to a mammal whereupon, e.g., the in vivo IL-27 or IL-35 production in the mammal is increased to artificially high levels, and autoimmunity is thereby suppressed in the mammal. IL-27 and IL-35 are rapidly cleared in vivo, however, the administration of the isolated population allows for sustained IL-27 or IL-35 administration in vivo. This provides distinct advantages over therapies that may rely upon, e.g., direct administration of IL-27 or IL-35.

IL-27 and IL-35 are two immune-suppressive members of the IL-12 family of cytokines. Although IL-35 or IL-27 show substantial promise in suppressing autoimmune diseases, a major disadvantage of using cytokines as biologics, especially heterodimeric cytokines, is their relatively short half-life, transient biological activities, and unpredictable pharmacokinetic characteristics. Another disadvantage relates to the issue of dosing. Because association of the IL-35 or IL-27 subunit proteins is not strong (non-covalent), IL-35 and IL-27 subunit proteins readily dissociate, thereby making it difficult to ascertain the effective dose of bioactive p35:Ebi3 or p28:Ebi3 heterodimer administered or required to ameliorate disease. Therapeutic use of exosomes of the invention provides several therapeutic advantages over the use of biologics such as IL-10, IL-27 or IL-35, which are the most effective cytokines produced by Breg or Treg cells. The use of exosomes in therapy has advantages over Breg therapy, including, for example: (1) the exosomes contain both subunits of IL-27 (IL27p28/Ebi3) or IL-35 (IL12p35/Ebi3) compartmentalized in a vesicle, obviating the dosing issue of ascertaining the amount of bioactive IL-35 or IL-27 administered, and (2) due to the small size of exosomes, the exosomes can be utilized to deliver IL-27 or IL-35 to CNS tissues to cross the BBB or BOB.

The term “autoimmunity,” as used herein, refers to the failure of an organism (e.g., a mammal, such as a human or mouse) to recognize its own constituent parts as self, which results in an immune response against the organism's own cells and tissues. In other words, autoimmunity is an adaptive immune response directed against “self” antigens and is marked by the production of proinflammatory cytokines that mediate pathology by damaging host tissues or by production of “autoantibodies” that can cause complement mediated diseases.

“Autoimmune disease” refers to any one of a group of diseases or disorders in which tissue injury is associated with a humoral and/or cell-mediated immune response to body constituents or, in a broader sense, an immune response to self. The pathological immune response may be systemic or organ specific. For example, the immune response directed against self may affect joints, skin, the brain, the myelin sheath that protects neurons, the kidneys, the liver, the pancreas, the thyroid, the adrenals, the eyes (e.g., uveitis), and ovaries. Immune complex formation plays a role in the etiology and progression of autoimmune disease. Increased immune complex formation correlates with the presence of antibodies directed to self (autoantibodies). The presence of autoantibodies can contribute to tissue inflammation either as part of an immune complex or unbound to antigen (free antibody). In some autoimmune diseases, the presence of free autoantibody contributes significantly to disease pathology. Another aspect of the etiology and progression of autoimmune disease is the role of proinflammatory cytokines. Under normal circumstances, proinflammatory cytokines such as tumor necrosis factor-α (TNF-α) and interleukin-1 (IL-1) play a protective role in the response to infection and cellular stress. However, the pathological consequences which result from chronic and/or excessive production of TNF-α and IL-1 are believed to underlie the progression of many autoimmune diseases such as rheumatoid arthritis, Crohn's disease, inflammatory bowel disease, uveitis, and psoriasis. Other proinflammatory cytokines involved in autoimmune disease include interleukin-6, interleukin-8, and granulocyte-macrophage colony stimulating factor (see, e.g., U.S. Pat. No. 8,080,555).

The inventive exosome populations and methods can be used to suppress autoimmunity associated with any autoimmune disease. There are more than 80 autoimmune diseases known in the art, examples of which include multiple sclerosis (MS), insulin-dependent diabetes mellitus, systemic lupus erythematosus (SLE), psoriasis, autoimmune hepatitis, thyroiditis, insulitis, uveitis, orchitis, myasthenia gravis, idiopathic thrombocytopenic purpura, inflammatory bowel diseases (e.g., Crohn's disease and ulcerative colitis), encephalomyelitis, systemic autoimmune diseases (e.g., rheumatoid arthritis (RA), scleroderma, and juvenile arthritis).

Autoimmunity is “suppressed” if one or more symptoms of an autoimmune disease is reduced or alleviated in a mammal (e.g., a human) affected by an autoimmune disease. Improvement, worsening, regression, or progression of a symptom may be determined by any objective or subjective measure, many of which are known in the art. A person of ordinary skill in the art will appreciate that the symptoms of autoimmune diseases vary based on the disease and location of the abnormal immune response. Symptoms that are common to several autoimmune diseases include, for example, fatigue, muscle and/or joint pain, muscle weakness, fever, swollen glands, inflammation, susceptibility to infections, weight loss or gain, allergies, digestive problems, blood pressure changes, and vertigo.

The inventive exosome population and methods can be used to decrease or suppress inflammation in the pancreas.

The inventive exosome population and methods can be used to decrease or suppress the symptoms of age-related macular degeneration (AMD).

As used herein, the terms “treatment,” “treating,” and the like refer to obtaining a desired pharmacologic and/or physiologic effect.

Preferably, the pharmacologic and/or physiologic effect is therapeutic, i.e., the effect partially or completely cures a disease and/or adverse symptom attributable to the disease. To this end, the inventive method comprises administering a “therapeutically effective amount” of the isolated exosome population. A “therapeutically effective amount” refers to an amount effective, at dosages and for periods of time necessary, to achieve a desired therapeutic result. The therapeutically effective amount may vary according to factors such as the disease state, age, sex, and weight of the individual, and the ability of the exosome population to elicit a desired response in the individual.

Alternatively, the pharmacologic and/or physiologic effect may be prophylactic, i.e., the effect completely or partially prevents an autoimmune disease or symptom thereof. In this respect, the inventive method comprises administering a “prophylactically effective amount” of the isolated exosome population to a mammal that is predisposed to, or otherwise at risk of developing, an autoimmune disease. A “prophylactically effective amount” refers to an amount effective, at dosages and for periods of time necessary, to achieve a desired prophylactic result (e.g., prevention of disease onset or prevention of disease flare-ups).

The isolated exosome population or composition comprising an isolated exosome population of the invention can be administered to a mammal using any suitable administration techniques, many of which are known in the art, including oral, intravenous, intraperitoneal, subcutaneous, pulmonary, transdermal, intramuscular, intranasal, buccal, sublingual, or suppository administration. The composition preferably is suitable for parenteral administration. The term “parenteral,” as used herein, includes intravenous, intramuscular, subcutaneous, rectal, vaginal, and intraperitoneal administration. More preferably, the composition is administered to a mammal using peripheral systemic delivery by intravenous, intraperitoneal, or subcutaneous injection.

Therapeutic or prophylactic efficacy can be monitored by periodic assessment of treated patients. For repeated administrations over several days or longer, depending on the condition, the treatment is repeated until a desired suppression of disease symptoms occurs. However, other dosage regimens may be useful and are within the scope of the invention. The desired dosage can be delivered by a single bolus administration of the composition, by multiple bolus administrations of the composition, or by continuous infusion administration of the composition.

A typical amount of cells administered to a mammal (e.g., a human) can be, for example, in the range of 500,000 to 100 million cells, although amounts below or above this exemplary range can be suitable in the context of the invention. For example, the daily dose of cells can be about 500,000 to about 50 million cells (e.g., about 5 million cells, about 15 million cells, about 25 million cells, about 35 million cells, about 45 million cells, or a range defined by any two of the foregoing values), preferably about 10 million to about 100 million cells (e.g., about 20 million cells, about 30 million cells, about 40 million, about 60 million cells, about 70 million cells, about 80 million cells, about 90 million cells, or a range defined by any two of the foregoing values), more preferably about 10 million cells to about 50 million cells (e.g., about 12 million cells, about 25 million cells, about 35 million cells, about 45 million cells, or a range defined by any two of the foregoing values).

The invention can be utilized in combination with other existing therapies for autoimmune diseases. For example, the exosome population of the invention can be administered in combination with immunosuppressive or immunomodulating agents or other anti-inflammatory agents for the treatment or prevention of an autoimmune disease, such as the autoimmune diseases disclosed herein. In this respect, the inventive method can be used in combination with disease-modifying anti-rheumatic drugs (DMARD) (e.g., gold salts, sulphasalazine, antimalarias, methotrexate, D-penicillamine, azathioprine, mycophenolic acid, cyclosporine A, tacrolimus, sirolimus, minocycline, leflunomide, and glucocorticoids), a calcineurin inhibitor (e.g., cyclosporin A or FK 506), a modulator of lymphocyte recirculation (e.g., FTY720 and FTY720 analogs), an mTOR inhibitor (e.g., rapamycin, 40-O-(2-hydroxyethyl)-rapamycin, CCI779, ABT578, AP23573, or TAFA-93), an ascomycin having immuno-suppressive properties (e.g., ABT-281, ASM981, etc.), corticosteroids, cyclophosphamide, azathioprene, methotrexate, leflunomide, mizoribine, mycophenolic acid, mycophenolate mofetil, 15-deoxyspergualine, or an immunosuppressive homologue, analogue or derivative thereof, immunosuppressive monoclonal antibodies (e.g., monoclonal antibodies to leukocyte receptors such as MEW, CD2, CD3, CD4, CD7, CD8, CD25, CD28, CD40. CD45, CD58, CD80, CD86, or their ligands), other immunomodulatory compounds, adhesion molecule inhibitors (e.g., LFA-1 antagonists, ICAM-1 or -3 antagonists, VCAM-4 antagonists, or VLA-4 antagonists), a chemotherapeutic agent (e.g., paclitaxel, gemcitabine, cisplatinum, doxorubicin, or 5-fluorouracil), anti-TNF agents (e.g. monoclonal antibodies to TNF such as infliximab, adalimumab, CDP870, or receptor constructs to TNF-RI or TNF-RII, such as ENBREL™ (Etanercept) or PEG-TNF-RI), blockers of proinflammatory cytokines, IL-1 blockers (e.g., KINERET™ (Anakinra) or IL-1 trap, AAL160, ACZ 885, and IL-6 blockers), chemokine blockers (e.g., inhibitors or activators of proteases), anti-IL-15 antibodies, anti-IL-6 antibodies, anti-CD20 antibodies, NSAIDs, and/or an anti-infectious agent.

The invention can be utilized in combination with administration of B cells that produce IL-35 or IL-27. The B cells that produce IL-35 (i35-Bregs) or IL-27 (i27-Bregs) can be administered sequentially (before or after) or simultaneously with the exosome population of the invention to a mammal.

The following include certain aspects of the invention:
1. An isolated population of exosomes comprising interleukin-27 (IL-27).
2. The isolated population of exosomes of aspect 1, wherein the population of exosomes further comprises IL-35.
3. An isolated population of exosomes comprising interleukin-35 (IL-35).
4. A method of preparing a population of exosomes comprising interleukin-27 (IL-27), the method comprising:
   (a) isolating CD19+B2 cells or B1a cells;
   (b) activating the isolated cells with a LPS or a BCR agonist to provide activated cells; and
   (c) isolating exosomes secreted from the activated cells.
5. A method of preparing a population of exosomes comprising interleukin-35 (IL-35), the method comprising:
   (a) isolating CD138+plasma cells;
   (b) activating the isolated cells with a LPS or a BCR agonist to provide activated cells; and
   (c) isolating exosomes secreted from the activated cells.
7. The population of exosomes of any one of aspects 1-3 for use in suppressing the immune system in a mammal.
8. The population of exosomes for the use according to aspect 7, wherein the population of exosomes comprises B cells that produce IL-35 or IL-27 to the mammal.
9. The population of exosomes for the use according to aspect 7 or 8, wherein the mammal has a disease.
10. The population of exosomes for the use according to any of one of aspects 7-9, wherein the mammal has an autoimmune disease.
11. The population of exosomes for the use according to aspect 10, wherein the autoimmune disease is a disease of the eye.
12. The population of exosomes for the use according to aspect 10, wherein the autoimmune disease is a disease of the central nervous system.
13. The population of exosomes for the use according to aspect 10, wherein the autoimmune disease is a disease of the brain.
14. The population of exosomes for the use according to aspect 10, wherein the autoimmune disease is uveitis.
15. The population of exosomes for the use according to aspect 10, wherein the autoimmune disease is encephalomyelitis.
16. The population of exosomes for the use according to any of one of aspects 7-9, wherein the mammal has multiple sclerosis.
17. The population of exosomes for the use according to any of one of aspects 7-9, wherein inflammation of the pancreas is suppressed.
18. The population of exosomes for the use according to aspect 7 or 8, wherein the mammal has received an allogeneic bone marrow or hematopoietic stem cell transplant.
19. The population of exosomes for the use according to aspect 7 or 8, wherein the mammal has received an allogeneic solid organ transplant.
20. The population of exosomes for the use according to aspect 18 or 19, wherein the mammal has graft-versus-host disease (GVHD).
21. The population of exosomes for the use according to any of one of aspects 7-9, wherein the mammal has age-related macular degeneration (AMD).

It shall be noted that the preceding are merely examples of embodiments. Other exemplary embodiments are apparent from the entirety of the description herein. It will also be understood by one of ordinary skill in the art that each of these embodiments may be used in various combinations with the other embodiments provided herein.

The following examples further illustrate the invention but, of course, should not be construed as in any way limiting its scope.

### EXAMPLE 1

This example demonstrates production and use of i35-Exosomes, in accordance with embodiments of the invention.

### Materials and Methods

### Mice

Six- to eight-week-old C57BL/6J mice (were purchased from Jackson Laboratory (Jackson Laboratory, Bar Harbor, ME, USA). Animal care and experimentation conformed to National Institutes of Health (NIH) guidelines and the experimental protocol was approved under NIH/NEI Animal Study Protocol (ASP) #NEI-597.

### Exosome Isolation and Characterization

Because i35-Bregs are mostly CD138+plasma cells, splenic B cells isolated by use of MicroBeads from Miltenyi-Biotec (130-121-301) are used. For generation of i35-Breg exosomes (i35-Exosomes), the plasma cells were stimulated with anti-IgM/anti-CD40 Abs for 72 h at low density (<10⁶/ml). Analysis of aliquots for p35 and Ebi3 expression by flow cytometry, routinely showed i35-Breg enrichment (>35%) under this culture condition as previously described (Wang et al., Nat. Med., 20: 633-41 (2014), incorporated by reference herein). Under this culture condition i35-Bregs or unstimulated CD19+control B cells do not die as verified by Vi-Cell XR (Viability Analyzer, Beckman Coulter, Indianapolis, IN, USA). Complete media with exosome-depleted FBS was used for exosome isolation from culture supernatants of control and i35-Breg enriched cultures using Exoquick TC reagent (System Biosciences, Palo Alto, CA, USA) following manufacturer's guidelines. Exosome size distribution was measured by Nanoparticle Tracking Analysis using the NanoSight system (NanoSight, Salisbury, United Kingdom) and expression of exosome markers or IL-35 subunit proteins were characterized by Western blotting.

### Western Blotting Analysis

Exosomes were lysed in RIPA buffer [10 mM Tris-Cl (pH 8.0), 1 mM EDTA, 1% of Triton X-100, 0.1% sodium deoxycholate, 0.1% SDS, 140 mM NaCl, and 1 mM PMSF] and lysates were incubated for 30 min on 4° C. After incubation, lysates were centrifuged at 14,000 rpm for 30 min and supernatants were harvested. Lysates (7 μg/lane) were fractionated on 4-12% gradient SDS-PAGE, and antibodies used were: CD63, CD9, HSP70 (System Biosciences #EXOAB-KIT-1), p35 (Santa Cruz Biotechnology, Dallas, TX, USA), and Ebi3 (Santa Cruz). After secondary antibodies reaction, signals were detected with LI-COR system (LI-COR Biosciences, Lincoln, NE, USA). Image studio software (LI-COR Biosciences) was used for data analysis.

### Immunoprecipitation

The i35-Exosome lysates were incubated with antibodies (4 μg of anti-Ebi3 or Normal IgG) overnight at 4° C. Next day, magnetic beads from Dynabeads Protein A Immunoprecipitation Kit (Thermo Fisher Scientific, Waltham, MA, USA) were incubated with lysates for 1 h at 4° C. and precipitated beads was washed and proteins were eluted and boiled for 10 min at 95° C. Samples were fractionated on 4-12% gradient SDS-PAGE and incubated with Ebi3 or p35 antibodies. After secondary antibodies reaction, signals were detected with the LI-COR system. Image studio software was used for data analysis.

### CFSE (Carboxyfluorescein Succinimidyl) Dilution Assay

For CFSE dilution assay, cells were cultured for 72 h using a commercially available CF SE Cell Proliferation kit (Molecular Probes, Inc., Eugene, OR, USA). Graphical display showing information about cells undergoing various rounds of cell division was obtained from FlowJo software. The threshold of cellular proliferation was determined based on analysis of unstimulated cells.

### ELISA

CD4+ cells were isolated from spleen and lymph nodes by MACS cell separation system (Miltenyi, Cologne, Germany). For T cell activation, cells were seeded on the plates precoated with 3 μg/ml of anti-CD3 antibodies and incubated with 1 μg/ml soluble anti-CD28 antibodies and PBS or exosomes. After 24 h, cytokines secreted in supernatant of the activated CD4+ T cells were analyzed by Multiplex ELISA (R&D Systems, Minneapolis, MN, USA).

### Experimental Autoimmune Uveitis (EAU)

EAU was induced by active immunization of C57BL/6J with IRBP651-670-peptide in a 0.2 ml emulsion (1:1 v/v with complete Freund's adjuvant (CFA)) containing Mycobacterium tuberculosis strainH37RA (2.5 mg/ml). Mice also received Bordetella pertussis toxin (1 μg/mouse) concurrently with immunization. Mice were matched by age and sex, and, for most experiments, 6-8 weeks mice were used (14 mice per group; n=14). Clinical disease was established and scored by fundoscopy and histology (Wang et al., Nat. Med., 20: 633-41 (2014), He et al., J. Autoimmun., 62:31-8(2015), and Oh et al., J. Immunol., 187: 3338-46 (2011), each of which is incorporated by reference herein).

### Histology

Eyes for histology were enucleated, fixed in 10% buffered formalin, and serially sectioned in the vertical pupillary-optic nerve plane. Specimens are then dehydrated through graded alcohol series, embedded in methacrylate, serial transverse sections (4 μm) cut, and stained with hematoxylin and eosin (H&E). Photographs of representative sections are taken on a photomicroscope.

### Fundoscopy

Funduscopic examinations were performed at day 15 and 17 after EAU induction. Fundus image was captured using Micron III retinal imaging microscope (Phoenix Research Labs) for small rodent or a modified Karl Storz veterinary otoendoscope coupled with a Nikon D90 digital camera, as previously described (Oh et al., J. Immunol., 187: 3338-46 (2011) and Paques et al., Invest. Ophthalmol. Vis. Sci., 48: 2769-74 (2007), each of which is incorporated by reference herein). To avoid a subjective bias was obviated by evaluating fundus photographs without knowledge of the mouse identity and by masked observers. At least six images (two posterior central retinal view, four peripheral retinal views) were taken from each eye by positioning the endoscope and viewing from superior, inferior, lateral, or medial fields, and each lesion was identified, mapped, and recorded. Clinical grading of retinal inflammation was as established (He et al., J. Autoimmun., 62: 31-8 (2015); Chan et al., J. Autoimmun., 3: 247-55 (1990), Xu et al., Exp. Eye Res., 87: 319-26 (2008); each of which is incorporated by reference herein).

### Optical Coherence Tomography (OCT)

Optical coherence tomography (OCT) is a non-invasive procedure that allows visualization of internal microstructure of various eye structures in living animals. Mice were immobilized using adjustable holder that allow for horizontal or vertical scan scanning and each scan was performed at least twice, with realignment each time. The dimension of the scan (in depth and transverse extent) was adjusted until the optimal signal intensity and contrast was achieved. Retinal thickness was measured from the central retinal area of all images obtained from both horizontal and vertical scans from the same eye, using the system software, and averaged. The method used to determine the retinal thicknesses in the system software was as described (He et al., J. Autoimmun., 62: 31-8 (2015) and Gabriele et al., Invest. Ophthalmol. Vis. Sci., 52: 2250-4 (2011), each incorporated by reference herein).

### Electroretinogram (ERG)

ERG measures changes in electrical potentials in response to light stimulation of the retina and is used to identify gross physiologic changes pathognomonic visual function defects. Before ERG recordings, mice are dark-adapted overnight, and experiments performed under dim red illumination. ERG is recorded on anesthetized mice using an electroretinography console that generates and controls the light stimulus. Dark adapted ERG is recorded with single-flash delivered in a Ganzfeld dome and a reference electrode (gold wire) is placed in the mouth, and a ground electrode (subcutaneous stainless steel needle) is positioned at the base of the tail. Signals are differentially amplified and digitized. Amplitudes of the major ERG components (a- and b-wave) are measured by automated methods (He et al., J. Autoimmun., 62: 31-8 (2015), incorporated by reference herein).

### Flow Cytometery

For intracellular cytokine detection, cells were restimulated for 4 h with PMA (20 ng/ml)/ionomycin (1 μM). GolgiStop was added in the last hour, and intracellular cytokine staining was performed using BD Biosciences Cytofix/Cytoperm kit as recommended (BD Pharmingen, San Diego, CA, USA). FACS analysis was performed on a MACSQuant analyzer (Miltenyi Biotec, San Diego, CA, USA) using protein-specific monoclonal antibodies and corresponding isotype control Abs (BD Pharmingen, San Diego, CA, USA) as described previously (Dambuza et al., Nat. Commun., 8: 719 (2017), incorporated by reference herein). FACS analysis was performed on samples stained with mAbs conjugated with fluorescent dyes, and each experiment was color-compensated. Dead cells were stained with dead cell exclusion dye (Fixable Viability Dye eFluorR 450; eBioscience, San Diego, CA, USA), and live cells were subjected to side scatter and forward scatter analysis. Quadrant gates were set using isotype controls with <0.2% background.

### Statistical Analysis

Statistical analyses were performed by independent two-tailed Students's t-test. The data are presented as mean±SEM.

### Results

i35-Breg-Derived Exosomes Suppressed CD4+T Cell Proliferation and INF-g Secretion In Vitro

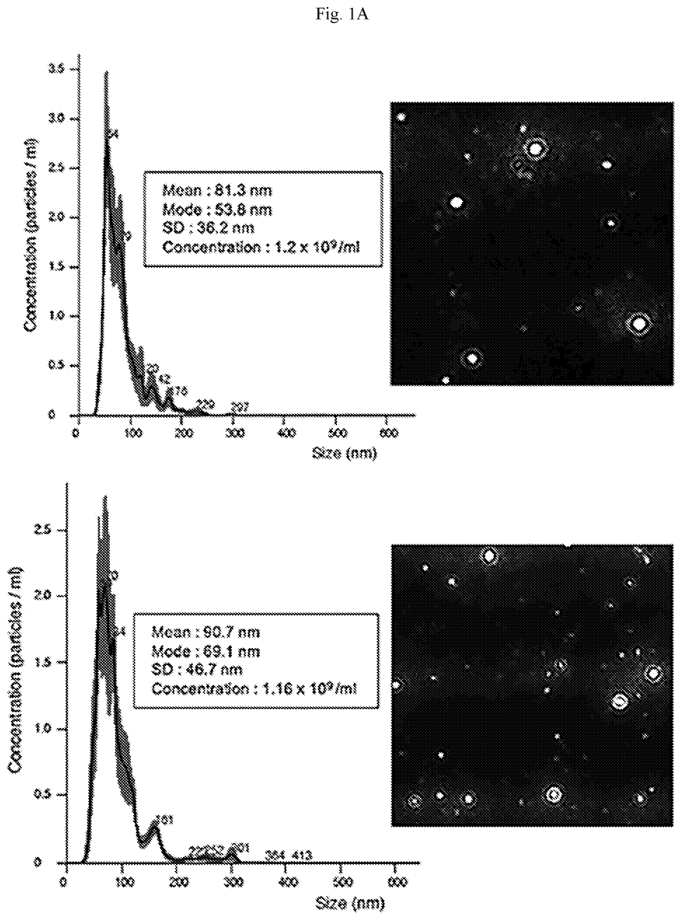

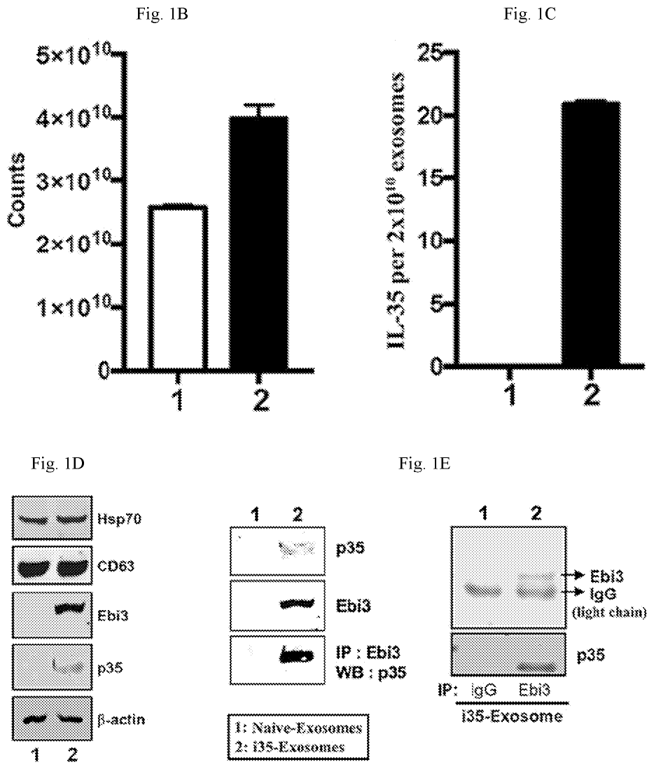

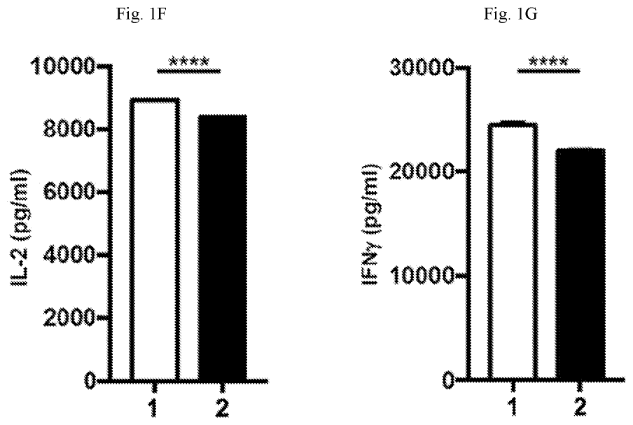

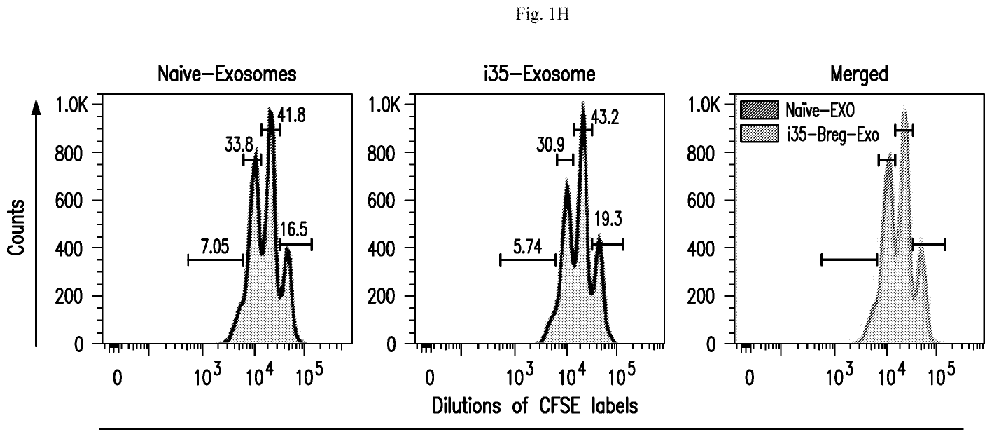

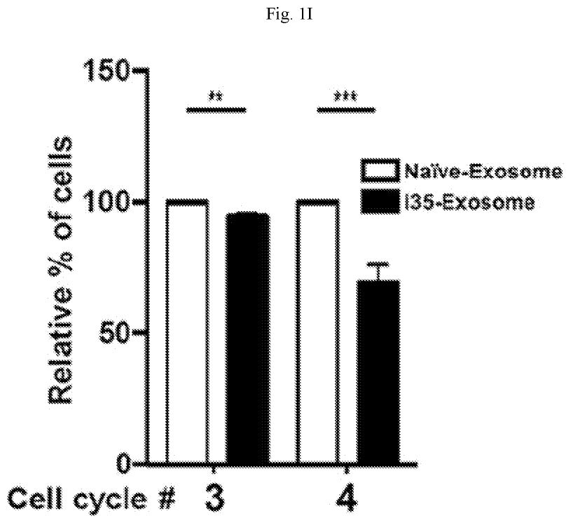

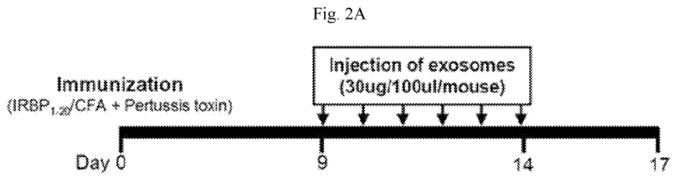

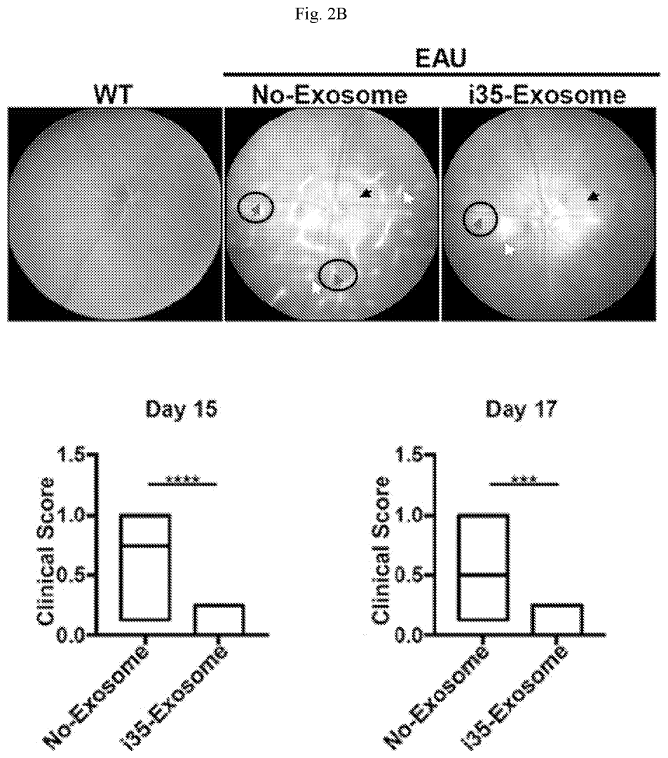

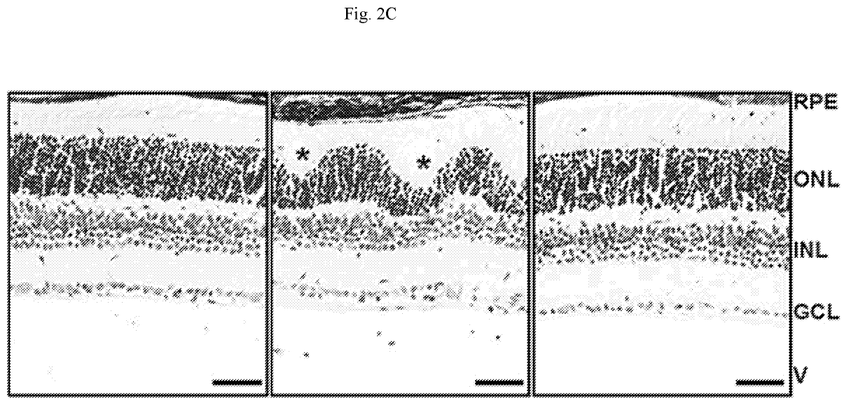

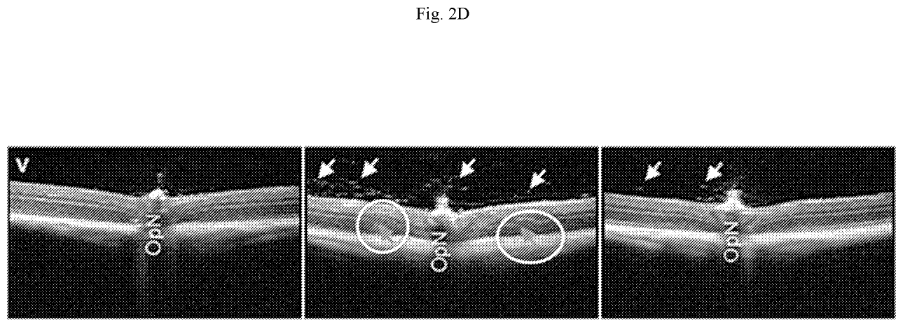

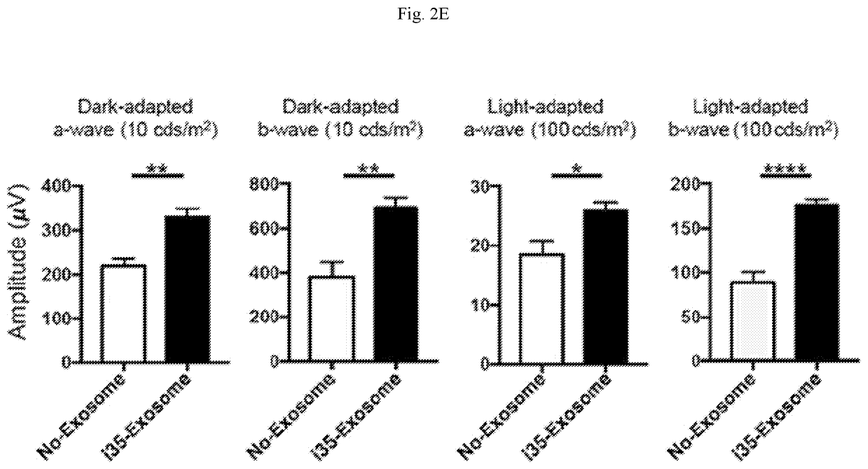

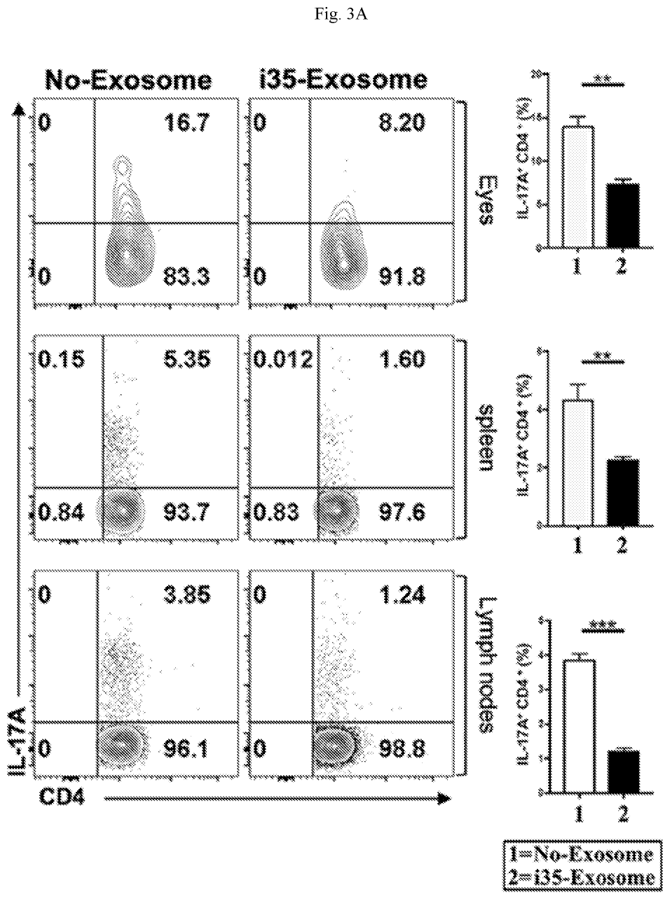

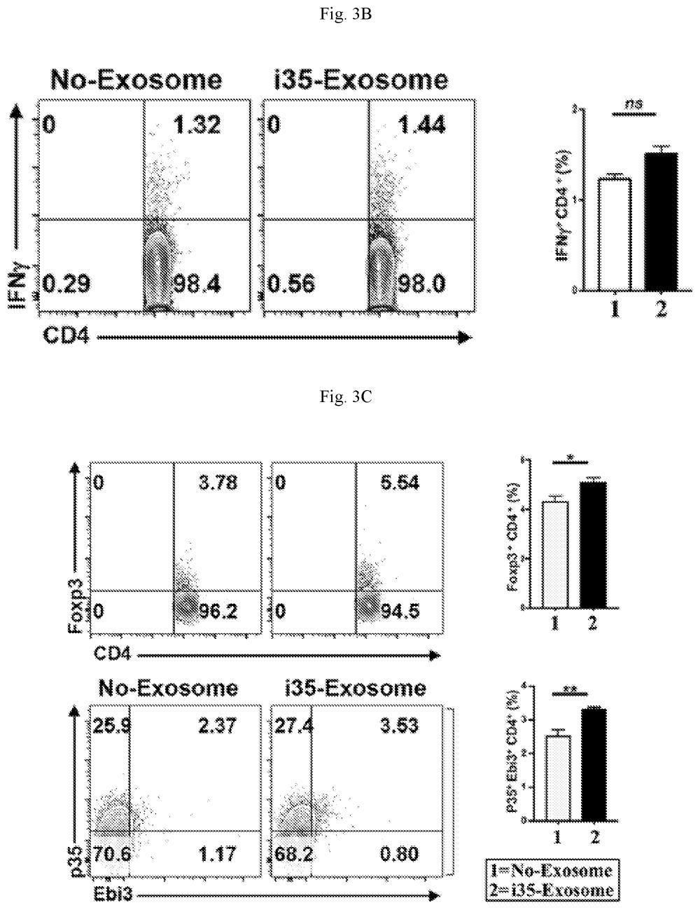

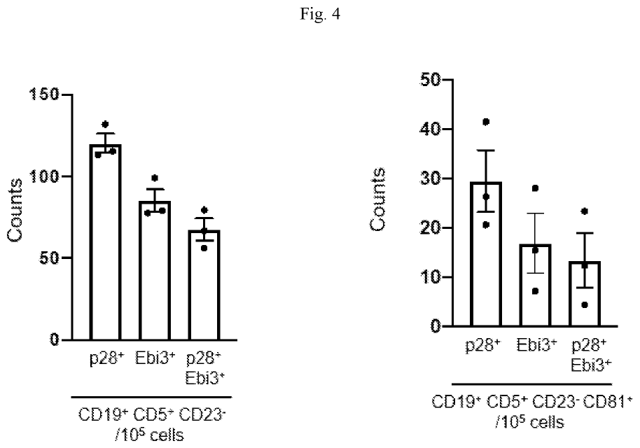

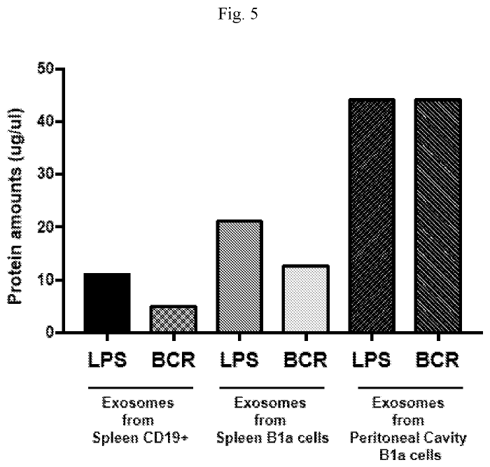

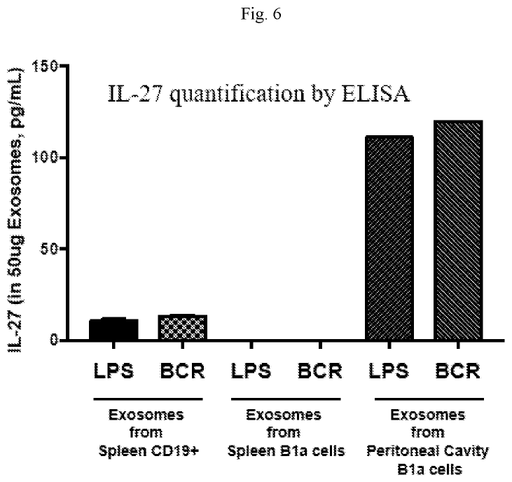

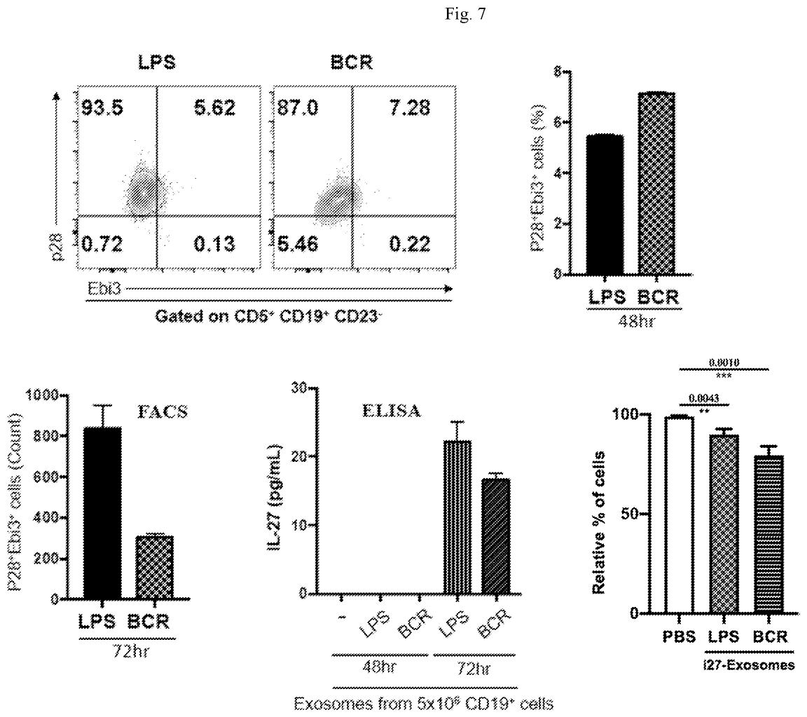

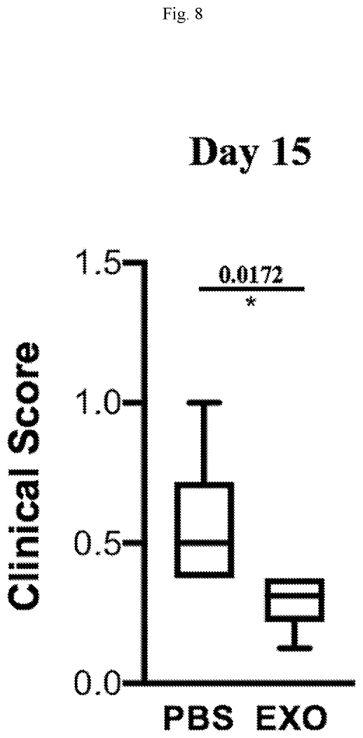

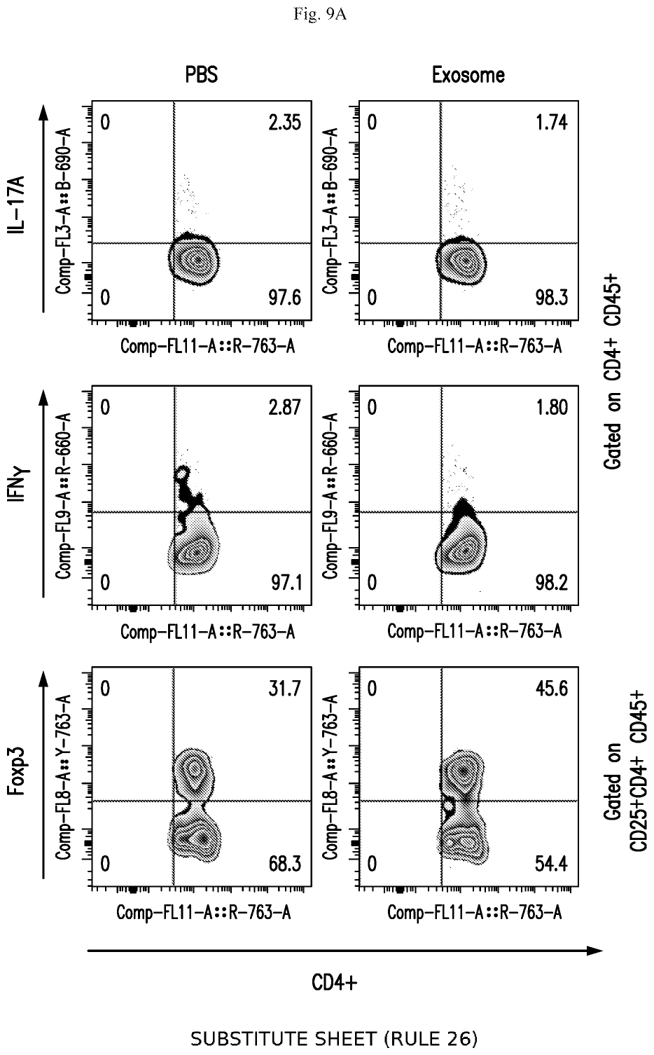

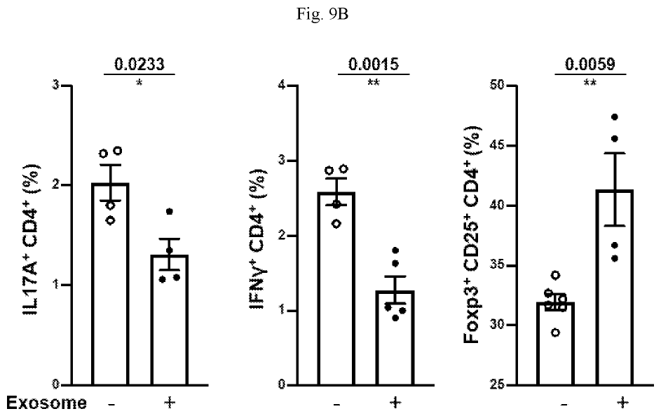

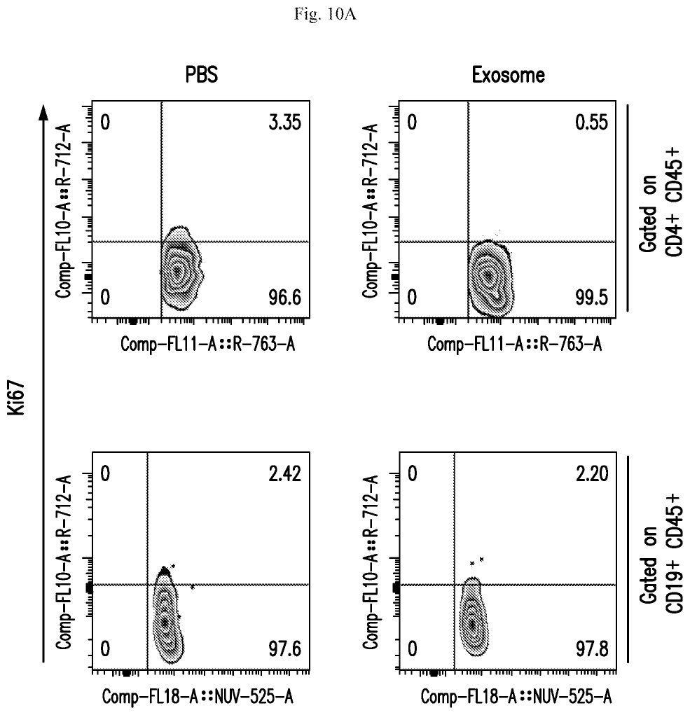

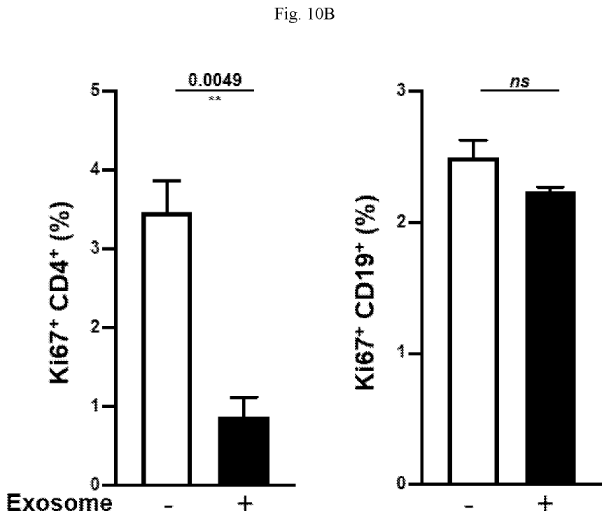

To examine whether B cells can secrete IL-35 containing exosomes, CD19+B cells (1×10⁶ cells) were seeded, stimulated with anti-IgM/anti-CD40 for 72 h, and pilot studies confirmed that IL-35-producing B cells (i35-Breg) were enriched in the culture (>35%) as previously described (Wang et al., Nat. Med., 20: 633-41 (2014) and Dambuza et al., Nat. Commun., 8: 719 (2017), each incorporated by reference herein). Control B cells were also cultured at 10⁶/ml, and under this low density culture condition, i35-Bregs or unstimulated CD19+control B cells do not die. Exosome enriched extracellular vesicles (EV) were isolated from the cell supernatant using ExoQuick exosome precipitation solution, and the Nanoparticle Tracking Analysis (NTA) method was used to determine particle size distribution of the exosomes which ranged from 50 to 150 nm for unstimulated CD19+B cells (Naïve-Exosome) and Breg-derived exosomes (i35-Exosomes) (FIG. 1A). Exosomes released in the culture were extracted from supernatants and exosome numbers was quantified using Exosome Quantitation Assay (System Biosciences). Average of 2.5×10¹⁰ and 4.0×10¹⁰ exosomes were secreted from unstimulated or stimulated CD19+B cells, respectively (FIG. 1B), and while exosomes from unstimulated B cells that did not produce IL-35 (Naïve-Exosomes), 2×10¹⁰ exosomes from i35-Exosomes were found to produce as much as 20 ng IL-35 (n=6) as determined by ELISA (FIG. 1C). Western blot analysis of lysates prepared from the exosomes showed that both Naïve-Exosome and i35-Exosomes expressed the canonical exosome-markers CD63 and Hsp70 and confirmed that the Naïve-Exosomes did not express IL-35 while the i35-Exosomes secreted both p35 and Ebi3 subunits that associate to produce the heterodimeric IL-35 cytokine (FIG. 1D).

To provide direct evidence that the i35-Exosomes produce the heterodimeric IL-35, reciprocal immunoprecipitation analysis was performed using antibodies specific to p35 and Ebi3. Precipitation of the extracts with the Ebi3 antibody and Western blot analysis using the p35 antibody confirmed the that the i35-Exosomes indeed produce the heterodimeric IL-35 (FIG. 1E). Immunoprecipitation data showing equivalent IgG light-chain band intensities indicates that equal amount of total lysate was used for lanes 1 and 2 (FIG. 1E, right panel).

Whether the i35-Exosomes could inhibit capacity of T cells to produce effector cytokines in response to TCR activation was investigated. Naïve CD4+ cells were isolated and purified from wild-type mice and stimulated for 3 days in medium containing anti-CD3/anti-CD28 antibodies and Naïve-Exosome or i35-Exosome (1.27×10¹⁰ exosomes). Analysis of supernatant by ELISA assay showed that compared to cultures that received Naïve-Exosomes, i35-Exosome suppressed TCR-mediated secretion of IL-2 (FIG. 1F) and IFN-γ (FIG. 1G). The effects of i35-Exosomes on T cell proliferative response were also examined by the CFSE dilution assay. Significant inhibition of T cell proliferation by i35-Exosomes is consistent with the observed decrease of IL-2 and IFN-γ secretion (FIG. 1H), underscoring efficacy of i35-Exosome in suppressing pro-inflammatory response of T cell in vitro.

i35-Exosomes Suppressed Established Experimental Autoimmune Uveitis (EAU)

In view of the immune-suppressive effect of i35-Exosomes in vitro, whether i35-Exosomes can be used to treat mice with experimental autoimmune uveitis (EAU), a model of human uveitis, was investigated. EAU was induced in C57BL/6J mice by active immunization with an autoantigenic peptide derived from interphotoreceptor retinoid-binding protein (IRBP651-670) in CFA emulsion. Mice were treated with −2×10 10 exosomes (30 μg/mouse) on day 9 post-immunization and every day until day 14 post-immunization by retro-orbital injection and disease severity was assessed on day-17 post-immunization. The immunization and exosome treatment strategy are shown (FIG. 2A). Disease progression was monitored by fundoscopy, histology, optical coherence tomography, and electroretinography. Fundus and histology images of control mice (PBS) show severe inflammation characterized by blurred optic disc margins and enlarged juxtapapillary areas, papilledema, retinal vasculitis with moderate cuffing, vitreitis, retinal folds, substantial infiltration of inflammatory cells into the vitreous, choroiditis, and yellow-whitish retinal and choroidal infiltrates (FIGS. 2B, C). In contrast, images derived from fundus (FIG. 2B) or histological (FIG. 2C) analyses revealed mild EAU in eyes of mice treated with i35-Exosomes and clinical scores were significantly low compared to eyes of the untreated mice (FIG. 2B).

Optical coherence tomography (OCT) is a non-invasive procedure that allows visualization of internal microstructure of various eye structures in living animals and results of the OCT analysis revealed substantial accumulation of inflammatory cells in vitreous and optic nerve head of control untreated eyes compared to mice treated with i35-Exosomes (FIG. 2D). Inflammation of the retina induces changes in the electroretinogram(ERG) indicative of alterations in visual function, and it is assessed by recording changes in electrical potentials in response to light stimulation of the retina. ERG under light-adaptive stimuli reflects cone-driven signaling while the dark-adapted b-wave responses evaluate status of rod-driven signaling and lower a- and b-wave recordings are indicative of retinal pathology. EAU pathology is associated with defects in rod and cones attributed to attack of photoreceptor cells by inflammatory Th17 and/or Th1 cells. Significant increase of a-wave and b-wave amplitudes was observed in eyes with i35-Exosomes compared to the control eyes (FIG. 2E), suggesting that defects in cone and rod signaling functions in normal mouse with EAU was rescued in part by i35-Exosome treatment. The observed defects in cone and rod signaling functions and higher EAU pathological score in untreated mice with EAU, suggest that i35-Exosomes contributed to mechanisms that prevented the decrement of visual impairment observed in mice during EAU.

i35-Exosomes Suppress Th17 Responses During EAU by Inducing Expansion of Treg Cells

EAU is a T cell mediated intraocular inflammatory disease and retinal pathology results in part from cytotoxic effects of proinflammatory cytokines secreted by inflammatory cells recruited into the retina during EAU. As Th1 and Th17 are implicated in the etiology of EAU, whether mechanistic basis for the suppression of EAU in mice treated with i35-Exosomes derived from antagonistic effects on proinflammatory responses was investigated. EAU was induced in C57BL/6J mice and fundoscopic examination of the eyes established that the development uveitis by day 15 post-immunization. The mice were then sacrificed on day 17 post-immunization, and lymphocytes isolated from the retina, spleen, or lymph nodes were analyzed by the intracellular cytokine assay. Analysis of cells that infiltrate the eye during EAU revealed significant of proinflammatory cytokines secreted by inflammatory cells recruited into the retina during EAU and significant reduction of Th17 cells in eyes of mice treated with i35-Exosomes but not control mouse eyes (FIG. 3A). Similarly, the levels of Th17 cells in the spleen or lymph nodes were markedly diminished providing evidence that i35-Exosomes antagonize Th17 responses during EAU (FIG. 3A). Analysis of the levels of Th1 cells in eyes of the EAU mice did not reveal significant difference between i35-Exosomes-treated and untreated mice (FIG. 3B), suggesting that i35-Exosomes antagonized Th17 but not Th1 responses, consistent with previous reports showing that EAU pathology is mediated primarily by Th17 cells.

As regulatory B cells (Bregs) and regulatory T cells (Tregs) have been shown to suppress pro-inflammatory responses that mediate uveitis, whether i35-Exosome-mediated attenuation of EAU derived in part from expansion of regulatory cells was examined. Lymphocytes that infiltrate the eye during EAU were analyzed, and it was observed that Foxp3+ regulatory T cells and IL-35-producing regulatory T cells (iTR35) are significantly expanded (FIG. 3C).

### DISCUSSION

Uveitis is a diverse group of intraocular inflammatory diseases that includes birdshot retinochoroidopathy, Behcet's disease, ocular sarcoidosis and accounts for 10% of severe visual handicaps in the United States. The disease can occur in the front of the eye (anterior uveitis), back of the eye (posterior uveitis) or all over the eye (pan uveitis) and may be of infectious or autoimmune etiology. Conventional treatment includes topical or systemic administration of corticosteroids. Although steroids are effective therapy for uveitis, serious adverse effects preclude their prolonged use. Biologics such as interferons, Tac antibody (Daclizumab), TNF-α blockers as well as slow-release ocular implants containing IL-10 provide viable alternatives to steroids in the treatment of recalcitrant, blinding ocular inflammatory diseases. However, mechanisms underlying efficacy of these therapies have not been fully elucidated and considerable impetus is to develop alternative therapies such as biologics and cell-based therapies for uveitis. Regulatory B cells show substantial promise for cell therapy against autoimmune and neurodegenerative diseases. However, significant technical difficulties and labor intensive efforts required to manufacture sufficient quantities for therapeutic use remain major obstacles to be overcome before they can be brought to the clinic. Moreover, Bregs suppress inflammation or autoimmune diseases in Ag specific manner, restricting their suppressive effects to the specific autoantigen that elicits the disease.

i35-Breg-mediated suppression and amelioration of uveitis or encephalomyelitis in mouse models of human uveitis or multiple sclerosis is attributed to inhibitory effects of IL-35 secreted at inflammatory sites by i35-Bregs. Breg cells release exosomes that contain bioactive IL-35 (i35-Exosomes) and this may be an additional mechanism by which i35-Breg cells suppress inflammatory responses. EAU shares essential clinical features of human uveitis and provides a useful framework for evaluating therapies purported to suppress and/or ameliorate uveitis. EAU also shares essential immunopathogenic features with EAE, the animal model multiple sclerosis. Thus, the EAU model was utilized to demonstrate that i35-Bregs can be used to treat a CNS autoimmune disease. i35-Exosomes suppressed EAU and conferred protection from ocular pathology by inhibiting the expansion and trafficking of pathogenic Th17 cells into the retina. ERG data showed that i35-Exosome rescued mice from decrement of retinal function associated with uveitis, underscoring the neuroprotective effect of i35-Exosome. Of clinical importance, i35-Exosome is non-toxic and mitigates uveitis without inducing systemic allogeneic immune responses, suggesting that i35-Exosome may complement antiinflammatory agents currently used to treat uveitis.

### EXAMPLE 2

This example demonstrates production and use of i27-Exosomes, in accordance with embodiments of the invention.

B1a cells of mouse peritoneal cavity were isolated using a B1a isolation kit. One million cells were seeded in 1 mL media and stimulated with anti IgM/anti-CD40 for 72 hrs. Exosomes were isolated from supernatant using EXO Quick-Ultra (System Biosciences) and then measured using the Exosome Quantitation Assay kit (System Biosciences). Approximately 300 billion exosomes (40 μg) are released from stimulated B1a cells. FIG. 4 shows activated mouse B1a cells co-express IL-27 (p28+/Ebi3+) and CD81 on the cell surface.

B-1 or B-2 cells were isolated from the peritoneal cavity, spleen, or blood by use of kits purchased from commercial sources such as CD19 MicroBeads or B220 MicroBeads. B-1a cells were also positively selected with magnetic beads conjugated with B-1a-specific antibodies. Cells (1×10⁶) were activated with lipopolysaccharide (LPS) or B cell receptor (BCR) activation (anti-IgM antibody and anti-CD40 antibody) for 72 hr and exosomes were isolated from supernatants and quantified. The results are shown in FIG. 5.

IL-27 quantification by ELISA is shown in FIG. 6. Approximately 100 μg/ml of IL-27 was contained in 50 μg B1a exosomes.

FIG. 7 presents flow cytometry plots and bar graphs showing B-1 cells in the spleen also secrete IL-27 and inhibit T cell proliferation.

CD9, CD63, and CD81 and IL-27 (p28/Ebi3) expression co-localized on the surface membrane of activated B1a cells. Each of p35 and ebi3 are colocalized with exosomal marker CD81, but p35 and ebi3 are not colocalized, suggesting that B1a cells can release p35-containing exosomes or Ebi3-containing exosomes, but not IL-35 containing exosomes.

i27-Exosomes suppressed experimental autoimmune uveitis (EAU), as shown in FIG. 8. EAU was induced in black mice and treated with phosphate-buffered saline (control, 100 μl) or 30 μg exosomes (in 10011.1) on day 6 post-immunization and every day until day 10 post-immunization by retro-orbital injection. Mice treated with B1 a-exosomes were found to have attenuated severe inflammatory responses.

i27-Exosomes suppressed EAU by inhibiting pro-inflammatory responses (IL-17 and IFN-γ) while inducing the expansion of regulatory Treg cells, as shown in FIGS. 9A and 9B.

i27-Exosomes ameliorated uveitis by suppressing proliferation of uveitogenic T cells, as shown in FIGS. 10A and 10B.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An isolated population of exosomes comprising interleukin-27 (IL-27), interleukin-35 (IL-35), or a combination of IL-27 and IL-35.

2-3. (canceled)

4. A method of preparing a population of exosomes comprising interleukin-27 (IL-27), the method comprising:
(a) isolating CD19+B2 cells or B1a cells;
(b) activating the isolated cells with a LPS or a BCR agonist to provide activated cells; and
(c) isolating exosomes secreted from the activated cells.

5. A method of preparing a population of exosomes comprising interleukin-35 (IL-35), the method comprising:
(a) isolating CD138+plasma cells;
(b) activating the isolated cells with a LPS or a BCR agonist to provide activated cells; and
(c) isolating exosomes secreted from the activated cells.

6. (canceled)

7. A method for suppressing the immune system in a mammal comprising administering a population of exosomes according claim 1 to a mammal.

8. The method of claim 7, wherein the population of exosomes comprises B cells that produce IL-35 or IL-27.

9. The method of claim 7, wherein the mammal has a disease.

10. The method of claim 7, wherein the mammal has an autoimmune disease.

11. The method of claim 10, wherein the autoimmune disease is a disease of the eye, a disease of the central nervous system, uveitis, or encephalomyelitis.

12. (canceled)

13. The method of claim 10, wherein the autoimmune disease is a disease of the brain.

14-15. (canceled)

16. The method of claim 7, wherein the mammal has multiple sclerosis.

17. The method of claim 7, wherein inflammation of the pancreas is suppressed.

18. The method of claim 7, wherein the mammal has received an allogeneic bone marrow or hematopoietic stem cell transplant.

19. The method of claim 7, wherein the mammal has received an allogeneic solid organ transplant.

20. The method of claim 7, wherein the mammal has graft-versus-host disease (GVHD).

21. The method of claim 7, wherein the mammal has age-related macular degeneration (AMD).

22. The method of claim 7, wherein the mammal is human.
